(19) United States
(12) Patent Application Publication
(10) Pub. No.: US 2026/0200838 A1
(43) Pub. Date: Jul. 16, 2026

(54) METHODS OF MAKING FORMAMIDINIUM LEAD IODIDE

(71) Applicant: Minh Tu Nguyen, Morrisville, NC (US)

(72) Inventor: Minh Tu Nguyen, Morrisville, NC (US)

(73) Assignee: PY Materials, LLC, Morrisville, NC (US)

(21) Appl. No.: 19/019,337

(22) Filed: Jan. 13, 2025

(51) Int. Cl. C07C 257/14

(52) U.S. Cl. CPC C07C 257/14

(57) ABSTRACT

The present disclosure relates to methods for making formamidinium lead iodide (FAPbI3). The methods comprise preparing a reaction mixture in various ways, including: (i) preparing a lead(ii) iodide suspension, wherein at least 90% of lead(ii) iodide is not dissolved and remains in solid state, followed by introducing formamidinium iodide to the suspension; (ii) mixing lead(ii) iodide and formamidinium iodide, followed by heating and cooling the solid mixture, which is then used to form a suspension with a liquid; or (iii) mixing a first precursor, which contains lead(ii) ions; a second precursor, which contains formamidinium ions; a third precursor, which contains iodide ions; and a liquid. The methods further comprise of heating the reaction mixture to a temperature greater than or equal to 40° C. and less than or equal to 190° C., followed by isolating formamidinium lead iodide product from the supernatant, and washing the product with a liquid.

### BACKGROUND

Perovskite solar cells (PSCs) have emerged as a leading candidate for low-cost solar technology in recent years, thanks to their potential for high efficiency, affordability, and ease of production. Their performance has rapidly improved, in some cases surpassing or matching that of traditional silicon-based solar cells. These cells offer great promise for lightweight, flexible, and scalable solar energy solutions. Additionally, perovskite materials are attracting significant attention for their potential in a wide range of optoelectronic devices, owing to their unique optical and electronic properties. These materials are particularly well-suited for use in light-emitting diodes (LEDs), lasers, photodetectors, and displays. With exceptional light absorption, tunable bandgaps, and high charge-carrier mobility, perovskites are ideal for efficient light emission and detection. Their versatility also makes them easily integrated into various device structures, paving the way for the development of flexible, high-performance optoelectronic components. Formamidinium lead iodide (FAPbI3) has been extensively used in the production of high-performance solar cells and a range of optoelectronic devices. As such, there is a growing demand for adaptable synthesis methods and large-scale production of this photovoltaic active material.

### SUMMARY

According to some embodiments, a method for making formamidinium lead iodide (FAPbI3) comprises preparing a suspension of lead(ii) iodide (PbI2) in a liquid S1, wherein at least 90% of lead(ii) iodide is not dissolved and remains in solid state (step (1a)). The method further comprises introducing formamidinium iodide (FAI) to the suspension in step (1a) to form a reaction mixture, wherein the reaction mixture is a suspension (step (1b)). The method further comprises heating the reaction mixture in step (1b) to a temperature greater than or equal to 40° C. and less than or equal to 190° C. (step (1c)). Further still, the method comprises isolating formamidinium lead iodide product by separating the solid from the supernatant (step (1d)). Finally, the method comprises washing the formamidinium lead iodide product with a liquid S2 (step (1e)). According to some embodiments, the liquid S1 in step (1a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. According to some embodiments, heating the reaction mixture in step (1c) comprises applying a pressure; wherein the pressure is greater than or equal to 1 bar and less than or equal to 220 bar. According to some embodiments, heating the reaction mixture in step (1c) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor. According to some embodiments, the liquid S2 in step (1e) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

According to some embodiments, a method for making formamidinium lead iodide (FAPbI3) comprises mixing lead(ii) iodide (PbI2) and formamidinium iodide (FAI) in solid states to form a reaction mixture (step (2a)). The method further comprises heating the reaction mixture to a temperature greater than or equal to 20° C. and less than or equal to 160° C. (step (2b)). The method further comprises cooling the reaction mixture (step (2c)). Further still, the method comprises preparing a suspension by mixing the reaction mixture and a liquid S3 (step (2d))). Further still, the method comprises heating the suspension to a temperature greater than or equal to 40° C. and less than or equal to 190° C. (step (2e)). Furthermore, the method comprises isolating formamidinium lead iodide product by separating the solid from the supernatant (step (2f)). Finally, the method comprises washing the formamidinium lead iodide product with a liquid S4 (step (2g)). According to some embodiments, the liquid S3 in step (2d) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. According to some embodiments, heating the reaction mixture in step (2e) comprises applying a pressure; wherein the pressure is greater than or equal to 1 bar and less than or equal to 220 bar. According to some embodiments, heating the reaction mixture in step (2e) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor. According to some embodiments, the liquid S4 in step (2g) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

According to some embodiments, a method for making formamidinium lead iodide (FAPbI3) comprises preparing a reaction mixture (step (3a)), comprising: (i) a first precursor, which contains lead(ii) ions; (ii) a second precursor, which contains formamidinium ions; (iii) a third precursor, which contains iodide ions; and (iv) a liquid S5. The method further comprises heating the reaction mixture to a temperature greater than or equal to 40° C. and less than or equal to 190° C. (step (3b)). Further still, the method comprises isolating formamidinium lead iodide product by separating the solid from the supernatant (step (3c)). Finally, the method comprises washing the formamidinium lead iodide product with a liquid S6 (step (3d)). According to some embodiments, the counterions of lead(ii) ions in the first precursor comprise at least one of formate, acetate, benzoate, lactate, nitrate, or iodide. According to some embodiments, the counterions of formamidinium ions in the second precursor comprise at least one of formate, acetate, propanoate, butanoate, pentanoate, hexanoate, benzoate, oxalate, malonate, lactate, citrate, hydrogen citrate, dihydrogen citrate, nitrate, or iodide. According to some embodiments, the counterions of iodide ions in the third precursor comprise at least one of Li⁺, Na⁺, K⁺, H₃O⁺, NH₄₊or (CH₃)₄N⁺. According to some embodiments, the liquid S5 in step (3a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. According to some embodiments, heating the reactions mixture in step (3b) comprises applying a pressure; wherein the pressure is greater than or equal to 1 bar and less than or equal to 220 bar. According to some embodiments, heating the reactions mixture in step (3b) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor. According to some embodiments, the liquid S6 in step (3d) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

### BRIEF DESCRIPTION OF THE DRAWINGS

FIG. 1 is a flowchart for an example method 100 of making formamidinium lead iodide (FAPbI3).

FIG. 2 is a flowchart for an example method 200 of making formamidinium lead iodide (FAPbI3).

FIG. 3 is a flowchart for an example method 300 of making formamidinium lead iodide (FAPbI3).

### DETAILED DESCRIPTION OF PREFERRED EMBODIMENTS

The methods described in the present disclosure for making formamidinium lead iodide (FAPbI3) may be used to manufacture one or more aspects of perovskite solar cells. Other potential applications include, but are not limited to, light-emitting diodes (LEDs), lasers, photodetectors, phototransistors, gas sensors, batteries, field-effect transistors (FETs), photocatalysis, water splitting, and/or thermoelectric devices.

In some embodiments, the present disclosure provides methods of making formamidinium lead iodide based on suspension-based approaches, wherein a prepared mixture contains components in both solid state and liquid state. In some embodiments, the present disclosure provides methods of making formamidinium lead iodide involves heterogeneous mixtures, wherein a prepared mixture is a combination of liquids and/or solids, in which the components do not blend uniformly, leading to the formation of distinct layers.

In some embodiments, the present disclosure provides methods of making formamidinium lead iodide, wherein the formamidinium lead iodide products could be in cubic phase (black color), tetragonal phase (yellow color), or combinations thereof. In some embodiments, formamidinium lead iodide product may be treated in certain manners, including, but not limited to, heating, cooling, keeping in vacuum, or aging, to facilitate the phase transition from cubic phase to tetragonal phase or vice versa, i.e., from tetragonal phase to cubic phase. The formamidinium lead iodide product may be comprised of both cubic phase and tetragonal phase.

In some embodiments, the present disclosure provides methods of making formamidinium lead iodide, wherein the formamidinium lead iodide product comprise of crystalline powder. In some embodiments, the formamidinium lead iodide product comprise of single crystals, polycrystalline crystals, and/or combination thereof. In some embodiments, the formamidinium lead iodide crystals have a size between 1 and 1,000 microns, preferably between 10 and 100 microns. In some embodiments, the present disclosure provides methods of making formamidinium lead iodide could result in formamidinium lead iodide crystals with a size greater than or equal 0.1 micron and less than or equal to 1 micron. In some embodiments, the present disclosure provides methods of making formamidinium lead iodide could result in formamidinium lead iodide crystals with a size greater than or equal to 1 millimeter and less than or equal to 1 millimeter.

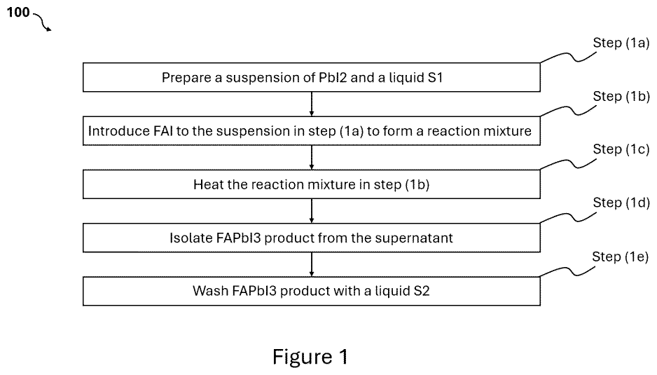

FIG. 1 illustrates an exemplary method 100 for making formamidinium lead iodide (FAPbI3). Step (1a) comprises preparing a suspension of lead(ii) iodide (PbI2) in a liquid S1, wherein at least 90% of lead(ii) iodide is not dissolved and remains in solid state. The liquid S1 in step (1a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. In some embodiments, the lead(ii) iodide used in step (1a) may have a purity between 91% and 99.99%. In some embodiments, preparing a suspension in step (1a) is carried out using at least one of mechanical, ultrasonic, vibration, pneumatic, ball-milling, and high-shear mixing methods. The method 100 continues at step (1b) with introducing formamidinium iodide (FAI) to the lead(ii) iodide suspension to form a reaction mixture, wherein the reaction mixture is a suspension. In some embodiments, step (1b) involves using at least one of mechanical, ultrasonic, vibration, ball-milling, and high-shear mixing methods. The method 100 then proceeds to step (1c) with heating the reaction mixture to a temperature greater than or equal to 40° C. and less than or equal to 190° C. In some embodiments, heating the reaction mixture in step (1c) is carried out in batch processing. In other embodiments, heating the reaction mixture in step (1c) is carried out in continuous processing, wherein the resulting mixture in step (1b) is continuously fed into a reactor; wherein the temperature of the reactor is kept at a temperature greater than or equal to 50° C. and less than or equal to 190° C.; and wherein continuously a product mixture comprising formamidinium lead iodide is removed from the reactor. In some embodiments, heating the reaction mixture in step (1c) is carried out with applying a pressure to the reaction mixture, wherein the pressure of the reaction mixture is adjusted to a value greater than or equal to 1 bar and less than or equal to 220 bar. The method 100 further continues with step (1d) with isolating formamidinium lead iodide product by separating the solid from the supernatant. In some embodiments, separating the formamidinium lead iodide solid in step (1d) may be achieved by any unit operation suitable for separating a solid, with examples including, but not limited to, decantation, centrifugation, filtration, and/or membrane separation. In some embodiments, separating the formamidinium lead iodide solid in step (1d) is carried out when the reaction mixture is cooled to room temperature. In other embodiments, separating the formamidinium lead iodide solid in step (1d) is carried out when the reaction mixture is not cooled to room temperature. Finally, the example method 100 concludes at step (1e) comprises washing the formamidinium lead iodide product with a liquid S2. In some embodiments, the liquid S2 in step (1e) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

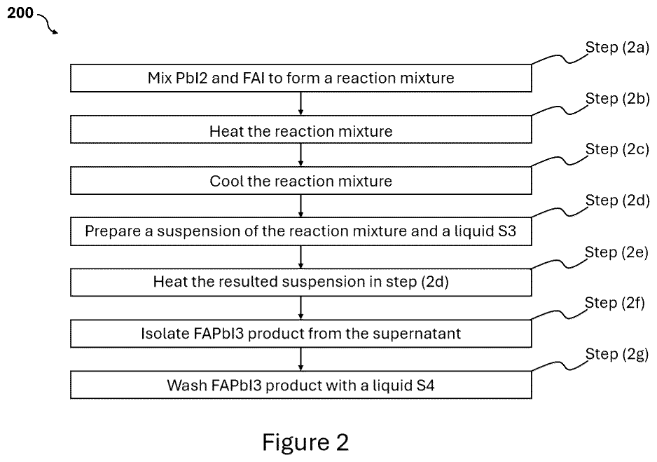

FIG. 2 illustrates an exemplary method 200 for making formamidinium lead iodide (FAPbI3). Step (2a) comprises mixing lead(ii) iodide (PbI2) and formamidinium iodide (FAI) in solid states to form a reaction mixture. In some embodiments, mixing in step (2a) is carried out using at least one of mechanical, ultrasonic, vibration, pneumatic, ball-milling, and high-shear mixing methods. In some embodiments, the lead(ii) iodide and/or formamidinium iodide used in step (1a) may have a purity between 91% and 99.99%. The method 200 continues at step (2b) with heating the reaction mixture to a temperature greater than or equal to 20° C. and less than or equal to 160° C. The method 200 then proceeds to step (2c) with cooling the reaction mixture. In some embodiments, cooling the reaction mixture in step (2c) comprises using air cooling, liquid cooling, evaporative cooling, thermoelectric cooling, and/or convection cooling. In some embodiments, cooling the reaction mixture in step (2c) comprises cooling the reaction mixture to room temperature. In other embodiments, cooling the reaction mixture in step (2c) comprises cooling the reaction mixture to a temperature greater than or equal to 30° C. and less than or equal to 140° C. The method 200 further continues with step (2d) with preparing a suspension by mixing the reaction mixture in step (2c) and a liquid S3. In some embodiments, mixing in step (2d)) is carried out using at least one of mechanical, ultrasonic, vibration, pneumatic, ball-milling, and high-shear mixing methods. In some embodiments, the liquid S3 in step (2d) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. Further still, the method 200 continues with step (2e) with heating the suspension to a temperature greater than or equal to 40° C. and less than or equal to 190° C. In some embodiments, heating the reaction mixture in step (2e) is carried out in batch processing. In other embodiments, heating the reaction mixture in step (2e) is carried out in continuous processing, wherein the resulting mixture in step (2d) is continuously fed into a reactor; wherein the temperature of the reactor is kept at a temperature greater than or equal to 50° C. and less than or equal to 190° C.; and wherein continuously a product mixture comprising formamidinium lead iodide is removed from the reactor. In some embodiments, heating the reaction mixture in step (2e) is carried out with applying a pressure to the reaction mixture, wherein the pressure of the reaction mixture is adjusted to a value greater than or equal to 1 bar and less than or equal to 220 bar. The method 200 further continues with step (2f) with isolating formamidinium lead iodide product by separating the solid from the supernatant. In some embodiments, separating the formamidinium lead iodide solid in step (2f) may be achieved by any unit operation suitable for separating a solid, with examples including, but not limited to, decantation, centrifugation, filtration, and/or membrane separation. In some embodiments, separating the formamidinium lead iodide solid in step (2f) is carried out when the reaction mixture is cooled to room temperature. In other embodiments, separating the formamidinium lead iodide solid in step (2f) is carried out when the reaction mixture is not cooled to room temperature. Finally, the example method 200 concludes at step (2g) comprises washing the formamidinium lead iodide product with a liquid S4. In some embodiments, the liquid S4 in step (2g) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

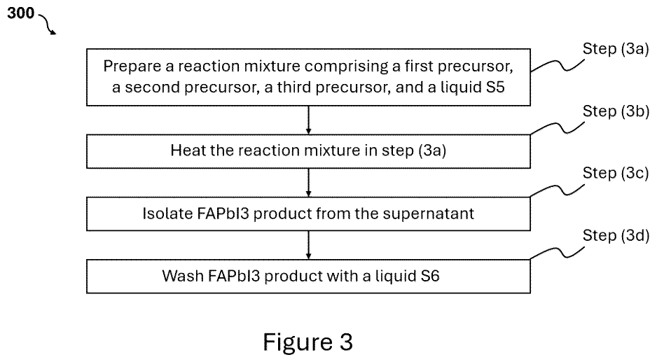

FIG. 3 illustrates an exemplary method 300 for making formamidinium lead iodide (FAPbI3). Step (3a) comprises preparing a reaction mixture comprising: (i) a first precursor, which contains lead(ii) ions; (ii) a second precursor, which contains formamidinium ions; (iii) a third precursor, which contains iodide ions; and (iv) a liquid S5. In some embodiments, preparing a reaction mixture in step (3a) is carried out using at least one of mechanical, ultrasonic, vibration, pneumatic, ball-milling, and high-shear mixing methods. In some embodiments, the counterions of lead(ii) ions in the first precursor in step (3a) comprise at least one of formate, acetate, benzoate, lactate, nitrate, or iodide. In some embodiments, the counterions of formamidinium ions in the second precursor in step (3a) comprise at least one of formate, acetate, propanoate, butanoate, pentanoate, hexanoate, benzoate, oxalate, malonate, lactate, citrate, hydrogen citrate, dihydrogen citrate, nitrate, or iodide. In some embodiments, the counterions of iodide ions in the third precursor in step (3a) comprise at least one of Li⁺, Na⁺, K⁺, H₃O⁺, NH₄⁺ or (CH₃)₄N⁺. In some embodiments, the liquid S5 in step (3a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof. The method 300 continues at step (3b) with heating the reaction mixture in step (3a) to a temperature greater than or equal to 40° C. and less than or equal to 190° C. In some embodiments, heating the reaction mixture in step (3b) is carried out in batch processing. In other embodiments, heating the reaction mixture in step (3b) is carried out in continuous processing, wherein the resulting mixture in step (3a) is continuously fed into a reactor; wherein the temperature of the reactor is kept at a temperature greater than or equal to 50° C. and less than or equal to 190° C.; and wherein continuously a product mixture comprising formamidinium lead iodide is removed from the reactor. In some embodiments, heating the reaction mixture in step (3b) is carried out with applying a pressure to the reaction mixture, wherein the pressure of the reaction mixture is adjusted to a value greater than or equal to 1 bar and less than or equal to 220 bar. The method 300 then proceeds to step (3c) with isolating formamidinium lead iodide product by separating the solid from the supernatant. In some embodiments, separating the formamidinium lead iodide solid in step (3c) may be achieved by any unit operation suitable for separating a solid, with examples including, but not limited to, decantation, centrifugation, filtration, and/or membrane separation. In some embodiments, separating the formamidinium lead iodide solid in step (3c) is carried out when the reaction mixture is cooled to room temperature. In other embodiments, separating the formamidinium lead iodide solid in step (3c) is carried out when the reaction mixture is not cooled to room temperature. Finally, the example method 300 concludes at step (3d) comprises washing the formamidinium lead iodide product with a liquid S6. In some embodiments, the liquid S6 in step (3d) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

While various embodiments have been presented in this disclosure, it should be recognized that the described systems and methods can be implemented in many other forms without deviating from the essence or scope of the disclosure. The provided examples are intended to be illustrative rather than limiting, and the intention is not to restrict the disclosure to the specific details provided. For instance, different elements or components may be merged or incorporated into another system, or certain features may be excluded or not implemented.

Additionally, the techniques, systems, subsystems, and methods described and depicted in various embodiments as distinct or separate may be combined or integrated with other systems, modules, techniques, or methods without altering the scope of the present disclosure. Items that are shown or discussed as being coupled, directly coupled, or communicating with each other may instead be indirectly coupled or communicate through an interface, device, or intermediate component, whether electrically, mechanically, or by other means. Other modifications, substitutions, and alterations can be identified by those skilled in the art and can be made without departing from the spirit and scope of the disclosure.

In addition, no restrictions are imposed on the construction or design details presented, except as defined in the claims below. It is clear that the illustrative embodiments may be altered or adapted, and all such variations fall within the scope and intent of the present invention. Specifically, any range of values (e.g., “from about a to about b,” or equivalently “from approximately a to b,” or “from approximately a-b”) disclosed herein is understood to encompass all subsets of that range, including every possible range within the broader one. Moreover, unless explicitly and clearly defined by the patentee, the terms used in the claims retain their plain, ordinary meaning.

1. A method for making formamidinium lead iodide, comprising the steps of:
(1a) preparing a suspension of lead(ii) iodide in a liquid S1, wherein at least 90% of lead(ii) iodide is not dissolved and remains in solid state;
(1b) introducing formamidinium iodide to the suspension to form a reaction mixture, wherein the reaction mixture is a suspension;
(1c) heating the reaction mixture to a temperature greater than or equal to 40° C. and less than or equal to 190° C.;
(1d) isolating formamidinium lead iodide product by separating the solid from the supernatant; and
(1e) washing the formamidinium lead iodide product with a liquid S2 .

2. The method of claim 1, wherein the liquid S1 in step (1a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof.

3. The method of claim 1, wherein heating the reaction mixture in step (1c) comprises applying a pressure; wherein the pressure is greater than or equal to 1 bar and less than or equal to 220 bar.

4. The method of claim 1, wherein heating the reaction mixture in step (1c) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor.

5. The method of claim 1, wherein the liquid S2 in step (1e) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

6. A method for making formamidinium lead iodide, comprising the steps of:
(2a) mixing lead(ii) iodide and formamidinium iodide in solid states to form a reaction mixture;
(2b) heating the reaction mixture to a temperature greater than or equal to 20° C. and less than or equal to 160° C.;
(2c) cooling the reaction mixture;
(2d)) preparing a suspension by mixing the reaction mixture and a liquid S3;
(2e) heating the suspension to a temperature greater than or equal to 40° C. and less than or equal to 190° C.;
(2f)) isolating formamidinium lead iodide product by separating the solid from the supernatant; and
(2g)) washing the formamidinium lead iodide product with a liquid S4.

7. The method of claim 6, wherein the liquid S3 in step (2d)) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof.

8. The method of claim 6, wherein heating the suspension in step (2e)) comprises applying a pressure; wherein the pressure is greater than or equal to 1 bar and less than or equal to 220 bar.

9. The method of claim 6, wherein heating the suspension in step (2e)) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor.

10. The method of claim 6, wherein the liquid S4 in step (2g)) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.

11. A method for making formamidinium lead iodide, comprising the steps of:
(3a) preparing a reaction mixture comprising: (i) a first precursor, which contains lead(ii) ions; (ii) a second precursor, which contains formamidinium ions; (iii) a third precursor, which contains iodide ions; and (iv) a liquid S5;
(3b) heating the reaction mixture to a temperature greater than or equal to 40° C. and less than or equal to 190° C.;
(3c) isolating formamidinium lead iodide product by separating the solid from the supernatant; and
(3d) washing the formamidinium lead iodide product with a liquid S6.

12. The method of claim 12, wherein the counterions of lead(ii) ions in the first precursor in step (3a) comprise at least one of formate, acetate, benzoate, lactate, nitrate, or iodide.

13. The method of claim 12, wherein the counterions of formamidinium ions in the second precursor in step (3a) comprise at least one of formate, acetate, propanoate, butanoate, pentanoate, hexanoate, benzoate, oxalate, malonate, lactate, citrate, hydrogen citrate, dihydrogen citrate, nitrate, or iodide.

14. The method of claim 12, wherein the counterions of iodide ions in the third precursor in step (3a) comprise at least one of Li⁺, Na⁺, K⁺, H₃O⁺, NH₄⁺ or (CH₃)₄N⁺.

15. The method of claim 12, wherein the liquid S5 in step (3a) is selected from the group consisting of water, methanol, ethanol, 1-propanol, 2-propanol, 1-butanol, 2-butanol, ethylene glycol, 2-methoxyethanol, 2-ethoxyethanol, propylene glycol, 1-methoxy-2-propanol, 1-ethoxy-2-propanol, glycerol, methyl lactate, ethyl lactate, propylene carbonate, 4-butyrolactone, acetone, 2-butanone, acetonitrile, acrylonitrile, N-methyl-2-pyrrolidone, dimethylformamide, dimethyl sulfoxide, formic acid, acetic acid, and combinations thereof.

16. The method of claim 12, wherein heating the reactions mixture in step (3b) comprises applying a pressure; wherein the pressure is greater than or equal to 1 bar and less than or equal to 220 bar.

17. The method of claim 12, wherein heating the reactions mixture in step (3b) comprises continuously feeding the suspension into a reactor; wherein the temperature of the reactor is kept at a temperature greater than or equal to 50° C. and less than or equal to 190° C.; and wherein continuously a product mixture comprising formamidinium lead iodide is removed from the reactor.

18. The method of claim 12, wherein the liquid S6 in step (3d) is selected from the group consisting of pentane, hexane, heptane, benzene, toluene, xylene, methyl acetate, ethyl acetate, diethyl ether, diisopropyl ether, tetrahydrofuran, 1,4-dioxane, dichloromethane, chloroform, acetone, 2-butanone, acetonitrile, acrylonitrile, and combinations thereof.
